(19) United States
(12) Patent Application Publication
(10) Pub. No.: US 2025/0221758 A1
(43) Pub. Date: Jul. 10, 2025

(54) CURVED THERMAL CUTTER ASSEMBLY AND METHOD FOR MANUFACTURING SAME

(71) Applicant: Covidien LP, Mansfield, MA (US)

(72) Inventors: William E. ROBINSON, IV, Boulder, CO (US); James D. ALLEN, Boulder, CO (US); Jake H. YOUNG, Lafayette, CO (US)

(73) Assignee: Covidien LP, Mansfield, MA (US)

(21) Appl. No.: 18/852,800

(22) PCT Filed: Mar. 31, 2023
(86) PCT No.: PCT/IB2023/053246

§ 371 Date: Sep. 30, 2024

Related U.S. Application Data
(60) Provisional application No. 63/325,809, filed on Mar. 31, 2022.

(51) Int. Cl. A61B 18/14; A61B 17/00

(52) U.S. Cl. CPC A61B 18/1445; A61B 17/00; A61B 2017/00526; A61B 2018/1452

(57) ABSTRACT

A jaw member for a surgical instrument includes a curved jaw housing supporting a similarly curved electrically conductive tissue treating surface having slot defined therein and extending therealong, the slot defining a curve substantially parallel to the curve of the tissue treating surface. A thermal cutter assembly is disposed within a portion of the slot, the thermal cutter assembly including a curved substrate configured to support a resistive element deposited thereon. The resistive element is adapted to connect to an energy source and is configured to thermally conduct heat to a portion of the substrate exposed from within the slot. The resistive element is composed of conductive material, a thickness of the conductive material is varied along a length of the resistive element to compensate for inconsistencies in thermal heating as a result of a deposition process

### FIELD

The present disclosure relates to surgical instruments and, more particularly, to electrosurgical instruments for sealing and cutting tissue, and methods of manufacturing same.

### BACKGROUND

A surgical forceps is a pliers-like instrument that relies on mechanical action between its jaw members to grasp, clamp, and constrict tissue. Electrosurgical forceps utilize both mechanical clamping action and energy to heat tissue to treat, e.g., coagulate, cauterize, or seal, tissue. Typically, once tissue is treated, the surgeon has to accurately sever the treated tissue. Accordingly, many electrosurgical forceps are designed to incorporate a knife that is advanced between the jaw members to cut the treated tissue. As an alternative to a mechanical knife, an energy-based tissue cutting element may be provided to cut the treated tissue using energy, e.g., thermal, electrosurgical, ultrasonic, light, or other suitable energy.

### SUMMARY

As used herein, the term “distal” refers to the portion that is being described which is further from a user, while the term “proximal” refers to the portion that is being described which is closer to a user. Further, to the extent consistent, any or all of the aspects detailed herein may be used in conjunction with any or all of the other aspects detailed herein.

Provided in accordance with aspects of the present disclosure is a jaw member for a surgical instrument which includes a curved jaw housing supporting a similarly curved electrically conductive tissue treating surface having slot defined therein and extending therealong, the slot defining a curve substantially parallel to the curve of the tissue treating surface. A thermal cutter assembly is disposed within a portion of the slot, the thermal cutter assembly including a curved substrate configured to support a resistive element deposited thereon. The resistive element is adapted to connect to an energy source and is configured to thermally conduct heat to a portion of the substrate exposed from within the slot. The resistive element is composed of a conductive material, a thickness of the conductive material is varied along a length of the resistive element to compensate for inconsistencies in thermal heating as a result of a deposition process.

In aspects in accordance with the present disclosure, the conductive material is selected from the group consisting of aluminum, copper, chromium, titanium, stainless steel, nickel, chrome, tin, platinum, zinc, palladium, gold, nichrome, and ferritic iron-chromium-aluminum alloys.

In aspects in accordance with the present disclosure, the conductive material includes a thickness in the range of about 0.1 micron to about 500 microns along the length of the substrate.

In aspects in accordance with the present disclosure, the conductive material includes multiple materials selected from the group consisting of aluminum, copper, chromium, titanium, stainless steel, nickel, chrome, tin, platinum, zinc, palladium, gold, nichrome, and ferritic iron-chromium-aluminum alloys, the total thickness of the multiple materials having a thickness in the range of about 1 micron to about 30 microns.

In aspects in accordance with the present disclosure, the conductive material includes a first thickness proximate a center of the substrate and a second thickness proximate an outer periphery of the substrate. In other aspects in accordance with the present disclosure, the first thickness of the conductive material is less than the second thickness of the conductive material. In yet other aspects in accordance with the present disclosure, the first thickness of the conductive material is greater than the second thickness of the conductive material.

In aspects in accordance with the present disclosure, the conductive material includes a first thickness proximate a center of the substrate and a second thickness proximate the proximal and distal ends of the substrate wherein the cross-sectional area of the resistive element along the substrate is substantially consistent resulting in uniform heating of the thermal cutting assembly at any point along a length thereof.

In aspects in accordance with the present disclosure, the deposition process includes at least one of sputtering, thermal evaporation, thermal spraying, cathodic arcing, pulsed laser deposition, electron beam deposition, electroless strike, electro-plating, or shadow masking.

Provided in accordance with aspects of the present disclosure is a jaw member for a surgical instrument which includes a curved jaw housing supporting a similarly curved electrically conductive tissue treating surface having slot defined therein and extending therealong, the slot defining a curve substantially parallel to the curve of the tissue treating surface. A thermal cutter assembly is disposed within a portion of the slot, the thermal cutter assembly including a curved substrate configured to support an insulator thereon. The insulator, in turn, is configured to receive a resistive element deposited thereon. The resistive element is encapsulated by an encapsulant, is adapted to connect to an energy source and is configured to thermally conduct heat to a portion of the substrate exposed from within the slot. The resistive element is composed of conductive material, a thickness of the conductive material is varied along a length of the resistive element to compensate for inconsistencies in thermal heating as a result of a deposition process. The cross-sectional area of the resistive element along the substrate is substantially consistent resulting in uniform heating of the thermal cutting assembly at any point along a length thereof.

In aspects in accordance with the present disclosure, the conductive material is selected from the group consisting of aluminum, copper, chromium, titanium, stainless steel, nickel, chrome, tin, platinum, zinc, palladium, gold, nichrome, and ferritic iron-chromium-aluminum alloys.

In aspects in accordance with the present disclosure, the conductive material includes a thickness in the range of about 0.1 micron to about 500 microns along the length of the substrate.

In aspects in accordance with the present disclosure, the conductive material includes a first thickness proximate a center of the substrate and a second thickness proximate an outer periphery of the substrate.

In aspects in accordance with the present disclosure, the encapsulant is at least partially thermally conductive and is also disposed on an opposite side of the substrate.

Provided in accordance with aspects of the present disclosure is a method for manufacturing a thermal cutter assembly for use with a curved jaw member of a surgical instrument which includes: disposing an insulator atop a curved substrate along a length thereof; depositing a conductive material atop the insulator along a length thereof to form a resistive element and varying the thickness of the conductive material along the curve to insure a uniform temperature gradient of the resistive element when activated along the length thereof; and encapsulating the resistive element with an encapsulant.

In aspects in accordance with the present disclosure, the deposition process includes one or more of sputtering, thermal evaporation, thermal spraying, cathodic arcing, pulsed laser deposition, electron beam deposition, electroless strike, electro-plating, or shadow masking.

In aspects in accordance with the present disclosure, the conductive material is selected from the group consisting of aluminum, copper, chromium, titanium, stainless steel, nickel, chrome, tin, platinum, zinc, palladium, gold, nichrome, and ferritic iron-chromium-aluminum alloys.

In aspects in accordance with the present disclosure, the conductive material includes a first thickness proximate a center of the substrate and a second thickness proximate an outer periphery of the substrate.

In aspects in accordance with the present disclosure, the method further includes encapsulating an opposite side of the substrate with the encapsulant. In other aspects in accordance with the present disclosure, the encapsulant is partially thermally conductive.

### BRIEF DESCRIPTION OF DRAWINGS

The above and other aspects and features of the present disclosure will become more apparent in view of the following detailed description when taken in conjunction with the accompanying drawings wherein like reference numerals identify similar or identical elements.

FIG. 1 is a perspective view of a shaft-based electrosurgical forceps provided in accordance with the present disclosure shown connected to an electrosurgical generator;

FIG. 2 is a perspective view of a hemostat-style electrosurgical forceps provided in accordance with the present disclosure;

FIG. 3 is a schematic illustration of a robotic surgical instrument provided in accordance with the present disclosure;

FIG. 4 is a perspective view of a distal end portion of the forceps of FIG. 1, wherein first and second jaw members of an end effector assembly of the forceps are disposed in a spaced-apart position exposing a thermal cutter assembly;

FIG. 5 is a perspective view of a distal end portion of the forceps of FIG. 1, wherein first and second jaw members of the end effector assembly of the forceps are disposed in a spaced-apart position and the thermal cutter assembly is separated therefrom exposing a slot defined in one of the jaw members;

FIG. 6A is a schematic view of the thermal cutter assembly in accordance with the present disclosure;

FIG. 6B is a schematic side view of the thermal cutter assembly in accordance with

the present disclosure;

FIGS. 7A-7D are various schematic views of the thermal cutter assembly with a resistive element deposited thereon in varying thicknesses along a length or a width thereof to provide uniform heating along the thermal cutter assembly during activation thereof; and

FIGS. 8A and 8B are various side views of the thermal cutter assembly with the resistive element shown exposed thereon illustrating the varying thicknesses along a length or a width thereof to provide uniform heating along the thermal cutter assembly during activation thereof.

### DETAILED DESCRIPTION

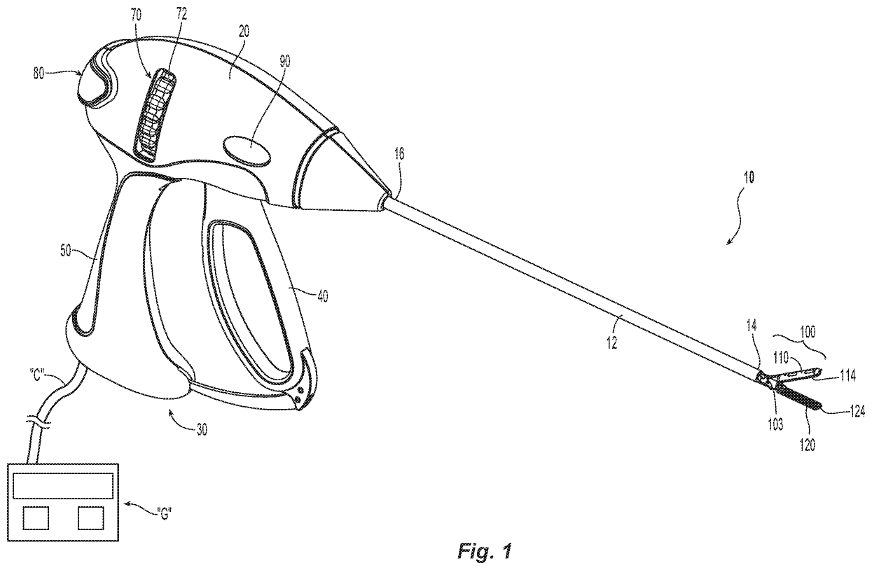

Referring to FIG. 1, a shaft-based electrosurgical forceps provided in accordance with the present disclosure is shown generally identified by reference numeral 10. Aspects and features of forceps 10 not germane to the understanding of the present disclosure are omitted to avoid obscuring the aspects and features of the present disclosure in unnecessary detail.

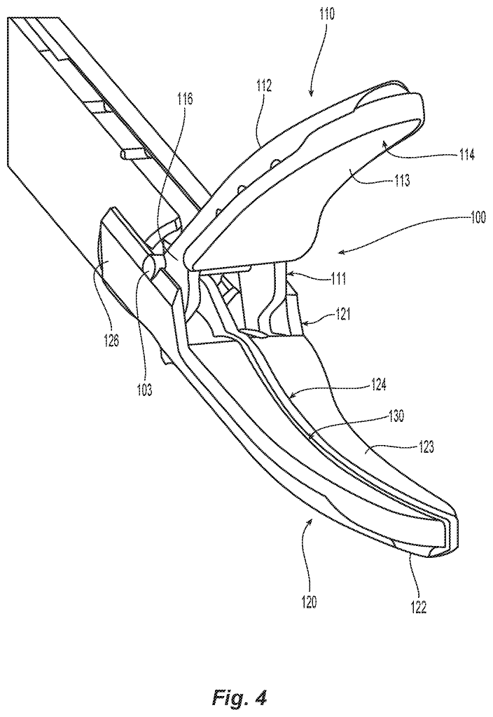

Forceps 10 includes a housing 20, a handle assembly 30, a rotating assembly 70, a first activation switch 80, a second activation switch 90, and an end effector assembly 100. As shown, end effector assembly 100 includes jaw members 110 and 120 configured for unilateral movement relative to one another. Bilateral movement of the jaw members 110, 120 is also envisioned. Forceps 10 further includes a shaft 12 having a distal end portion 14 configured to (directly or indirectly) engage end effector assembly 100 and a proximal end portion 16 that (directly or indirectly) engages housing 20. Forceps 10 also includes cable “C” that connects forceps 10 to an energy source, e.g., an electrosurgical generator “G.” Cable “C” includes a wire (or wires) (not shown) extending therethrough that has sufficient length to extend through shaft 12 in order to connect to one or both tissue-treating surfaces 114, 124 of jaw members 110, 120, respectively, of end effector assembly 100 (see FIG. 4) to provide energy thereto.

First activation switch 80 is coupled to tissue-treating surfaces 114, 124 (FIG. 4) and the electrosurgical generator “G” for enabling the selective activation of the supply of energy to jaw members 110, 120 for treating, e.g., cauterizing, coagulating/desiccating, and/or sealing, tissue. Second activation switch (e.g., thumb switch 90) is coupled to thermal cutter assembly 130 of jaw member 120 (FIG. 4) and the electrosurgical generator “G” for enabling the selective activation of the supply of energy to thermal cutter assembly 130 for thermally cutting tissue. Second activation switch 90 may be actuated via any finger, in-line with handle, footswitch, etc.

Alternatively, a single activation switch may be utilized wherein the generator “G” sequentially seals and then cuts with a single actuation of the switch, e.g., switch 80. A “seal” may be indicated by an audible tone from the generator “G” and after a short or programmable delay the forceps 10 (or the generator algorithm) transitions into a cut cycle or cut “mode”. Again a “cut” may be represented by a different tone from the generator “G” or from the forceps 10.

Handle assembly 30 of forceps 10 includes a fixed handle 50 and a movable handle 40. Fixed handle 50 is integrally associated with housing 20 and handle 40 is movable relative to fixed handle 50. Movable handle 40 of handle assembly 30 is operably coupled to a drive assembly (not shown) that, together, mechanically cooperate to impart movement of one or both of jaw members 110, 120 of end effector assembly 100 about a pivot 103 between a spaced-apart position and an approximated position to grasp tissue between tissue-treating surfaces 114, 124 of jaw members 110, 120. As shown in FIG. 1, movable handle 40 is initially spaced-apart from fixed handle 50 and, correspondingly, jaw members 110, 120 of end effector assembly 100 are disposed in the spaced-apart position. Movable handle 40 is depressible from this initial position to a depressed position corresponding to the approximated position of jaw members 110, 120. Rotating assembly 70 includes a rotation wheel 72 that is selectively rotatable in either direction to correspondingly rotate end effector assembly 100 relative to housing 20.

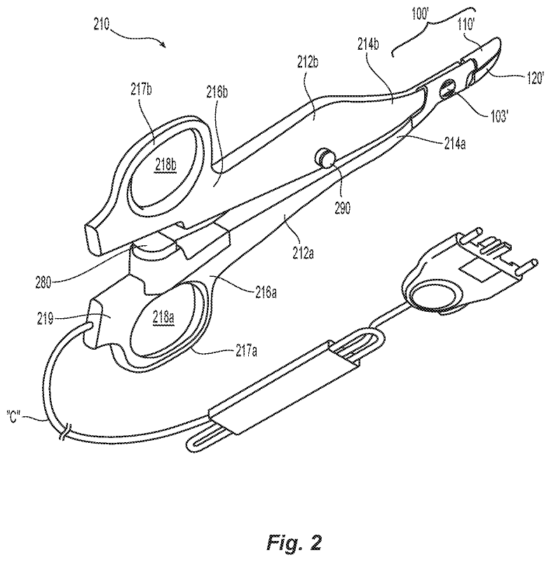

Referring to FIG. 2, a hemostat-style electrosurgical forceps provided in accordance with the present disclosure is shown generally identified by reference numeral 210. Aspects and features of forceps 210 not germane to the understanding of the present disclosure are omitted to avoid obscuring the aspects and features of the present disclosure in unnecessary detail.

Forceps 210 includes two elongated shaft members 212a, 212b, each having a proximal end portion 216a, 216b, and a distal end portion 214a, 214b, respectively. Forceps 210 is configured for use with an end effector assembly 100′ similar to end effector assembly 100 (FIG. 4). More specifically, end effector assembly 100′ includes first and second jaw members 110′, 120′ attached to respective distal end portions 214a, 214b of shaft members 212a, 212b. Jaw members 110′, 120′ are pivotably connected about a pivot 103′. Each shaft member 212a, 212b includes a handle 217a, 217b disposed at the proximal end portion 216a, 216b thereof. Each handle 217a, 217b defines a finger hole 218a, 218b therethrough for receiving a finger of the user. As can be appreciated, finger holes 218a, 218b facilitate movement of the shaft members 212a, 212b relative to one another to, in turn, pivot jaw members 110′, 120′ from the spaced-apart position, wherein jaw members 110′, 120′ are disposed in spaced relation relative to one another, to the approximated position, wherein jaw members 110′, 120′ cooperate to grasp tissue therebetween.

One of the shaft members 212a, 212b of forceps 210, e.g., shaft member 212b, includes a proximal shaft connector 219 configured to connect forceps 210 to a source of energy, e.g., electrosurgical generator “G” (FIG. 1). Proximal shaft connector 219 secures a cable “C” to forceps 210 such that the user may selectively supply energy to jaw members 110′, 120′ for treating tissue. More specifically, a first activation switch 280 (similar to activation switch 80 discussed above) is provided for supplying energy to jaw members 110′, 120′ to treat tissue upon sufficient approximation of shaft members 212a, 212b, e.g., upon activation of first activation switch 280 via shaft member 212a. A second activation switch 290 (similar to second activation switch 90 discussed above) disposed on either or both of shaft members 212a, 212b is coupled to the thermal cutter element (not shown, similar to thermal cutter assembly 130 of jaw member 120 (FIG. 4)) of one of the jaw members 110′, 120′ of end effector assembly 100′ and to the electrosurgical generator “G” for enabling the selective activation of the supply of energy to the thermal cutter assembly 130 for thermally cutting tissue.

Alternatively, a single activation switch may be utilized wherein the generator “G” sequentially seals and then cuts with a single actuation of the switch, e.g., switch 280. A “seal” may be indicated by an audible tone from the generator “G” and after a short or programmable delay the forceps 210 (or the generator algorithm) transitions into a cut cycle or cut “mode”. Again a “cut” may be represented by a different tone from the generator “G” or from the forceps 210.

Jaw members 110′, 120′ define a curved configuration wherein each jaw member is similarly curved laterally relative to a longitudinal axis of end effector assembly 100′. However, other suitable curved configurations including curvature towards one of the jaw members 110, 120′ (and thus away from the other), multiple curves with the same plane, and/or multiple curves within different planes are also contemplated. Jaw members 110, 120 of end effector assembly 100 (FIG. 1) may likewise be curved according to any of the configurations noted above or in any other suitable manner.

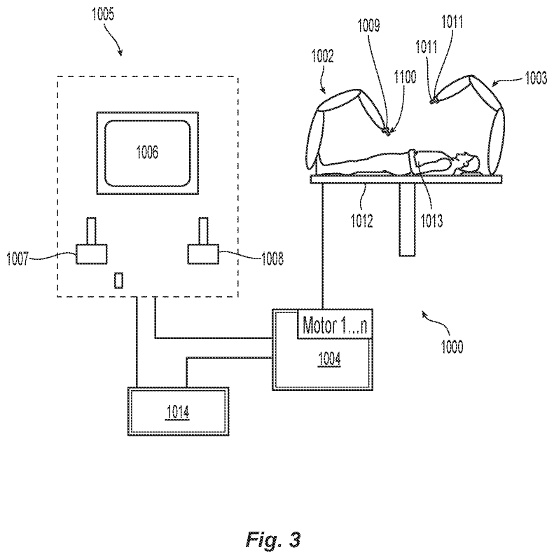

Referring to FIG. 3, a robotic surgical instrument provided in accordance with the present disclosure is shown generally identified by reference numeral 1000. Aspects and features of robotic surgical instrument 1000 not germane to the understanding of the present disclosure are omitted to avoid obscuring the aspects and features of the present disclosure in unnecessary detail.

Robotic surgical instrument 1000 includes a plurality of robot arms 1002, 1003; a control device 1004; and an operating console 1005 coupled with control device 1004. Operating console 1005 may include a display device 1006, which may be set up in particular to display three-dimensional images; and manual input devices 1007, 1008, by means of which a surgeon may be able to telemanipulate robot arms 1002, 1003 in a first operating mode. Robotic surgical instrument 1000 may be configured for use on a patient 1013 lying on a patient table 1012 to be treated in a minimally invasive manner. Robotic surgical instrument 1000 may further include a database 1014, in particular coupled to control device 1004, in which are stored, for example, pre-operative data from patient 1013 and/or anatomical atlases.

Each of the robot arms 1002, 1003 may include a plurality of members, which are connected through joints, and an attaching device 1009, 1011, to which may be attached, for example, an end effector assembly 1100, 1200, respectively. End effector assembly 1100 is similar to end effector assembly 100 (FIG. 4), although other suitable end effector assemblies for coupling to attaching device 1009 are also contemplated. End effector assembly 1200 may be any end effector assembly, e.g., an endoscopic camera, other surgical tool, etc. Robot arms 1002, 1003 and end effector assemblies 1100, 1200 may be driven by electric drives, e.g., motors, that are connected to control device 1004. Control device 1004 (e.g., a computer) may be configured to activate the motors, in particular by means of a computer program, in such a way that robot arms 1002, 1003, their attaching devices 1009, 1011, and end effector assemblies 1100, 1200 execute a desired movement and/or function according to a corresponding input from manual input devices 1007, 1008, respectively. Control device 1004 may also be configured in such a way that it regulates the movement of robot arms 1002, 1003 and/or of the motors.

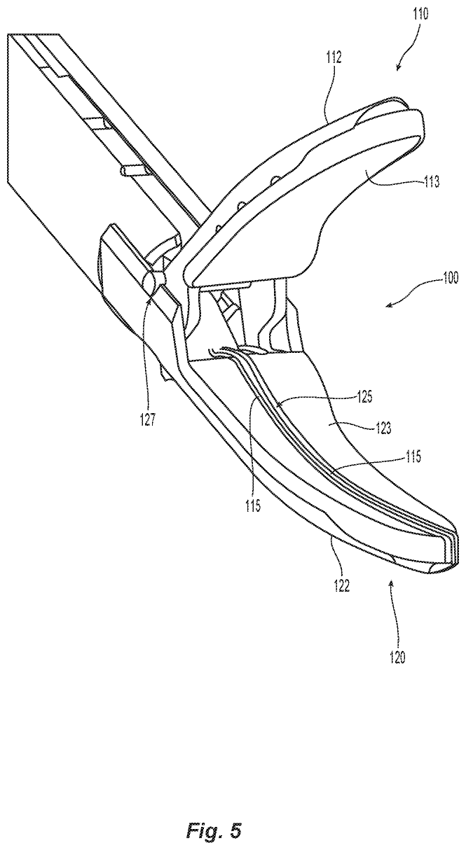

Turning to FIGS. 4-5, end effector assembly 100, as noted above, includes first and second jaw members 110, 120. Each jaw member 110, 120 may include a structural frame 111, 121, a jaw housing 112, 122, and a tissue-treating plate 113, 123 defining the respective tissue-treating surface 114, 124 thereof. Alternatively, only one of the jaw members, e.g., jaw member 120, may include structural frame 121, jaw housing 122, and tissue-treating plate 123 defining the tissue-treating surface 124. In such embodiments, the other jaw member, e.g., jaw member 110, may be formed as a single unitary body, e.g., a piece of conductive material acting as the structural frame 111 and jaw housing 112 and defining the tissue-treating surface 114.

An outer surface of the jaw housing 112, in such embodiments, may be at least partially coated with an electrically insulative material or may remain exposed. In embodiments, tissue-treating plates 113, 123 may be deposited onto jaw housings 112, 122 or jaw inserts (not shown) disposed within jaw housings 112, 122, e.g., via sputtering. Alternatively, tissue-treating plates 113, 123 may be pre-formed and engaged with jaw housings 112, 122 and/or jaw inserts (not shown) disposed within jaw housings 112, 122 via, for example, overmolding, adhesion, mechanical engagement, etc. Other methods of depositing the tissue-treating plates 113, 123 onto the jaw inserts are described in detail below.

Referring in particular to FIGS. 4 and 5, jaw member 110, as noted above, may be configured similarly as jaw member 120, may be formed as a single unitary body, or may be formed in any other suitable manner so as to define a structural frame 111 and a tissue-treating surface 114 opposing tissue-treating surface 124 of jaw member 120. Structural frame 111 includes a proximal flange portion 116 about which jaw member 110 is pivotably coupled to jaw member 120. In shaft-based or robotic embodiments, proximal flange portion 116 receives pivot 103 and which mounts atop flange 126 of jaw member 120 (FIG. 4) such that actuation of movable handle 40 (FIG. 1) or a robotic drive, pivots jaw member 110 about pivot 103 and relative to jaw member 120 between the spaced-apart position and the approximated position. However, other suitable drive arrangements are also contemplated, e.g., using cam pins and cam slots, a screw-drive mechanism, etc.

For the purposes of further describing one or both of the jaw members 110, 120 (and 210, 220), each jaw member 110, 120 may include a longitudinally-extending insulative member 115 defined within a slot 125 extending along at least a portion of the length of tissue-treating surfaces 114, 124 (FIG. 5). Insulative member 115 may be transversely centered on either or both tissue-treating surfaces 114, 124 or may be offset relative thereto. As explained in more detail below with respect to jaw member 120, insulative member 115 may house and electrically and/or thermally isolate the thermal cutter assembly 130 separately activatable to cut tissue upon activation thereof. Further, insulative member 115 may be disposed, e.g., deposited, coated, etc., on tissue-treating surface 114, 124, may be positioned within the channel or recess defined within tissue-treating surface 114, 124, or may define any other suitable configuration.

Additionally, insulative member 115 may be substantially (within manufacturing, material, and/or use tolerances) coplanar with each respective tissue-treating surface 114, 124 may protrude from each respective tissue-treating surface 114, 124, may be recessed relative to each respective tissue-treating surface 114, 124 or may include different portions that are coplanar, protruding, and/or recessed relative to tissue-treating surfaces 114, 124. Moreover, insulative member 115 and thermal cutter assembly 130 may be curvilinear to follow the configuration of the jaw members 110, 120. Insulative member 115 may be formed from, for example, ceramic, parylene, glass, nylon, PTFE, or other suitable material(s) (including combinations of insulative and non-insulative materials).

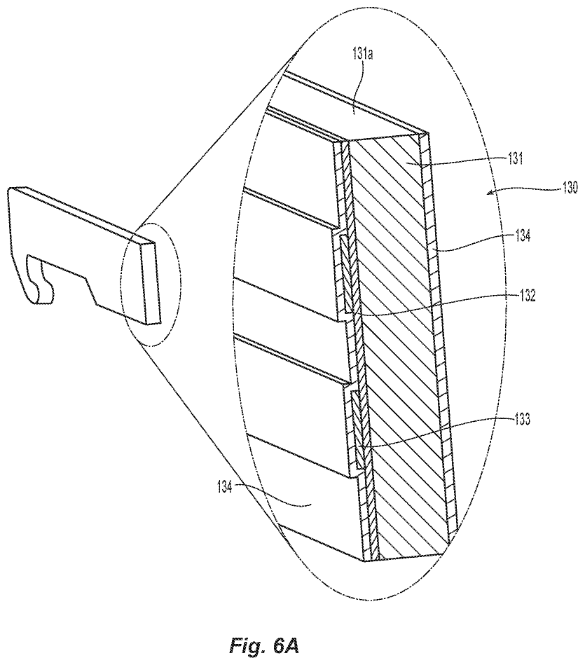

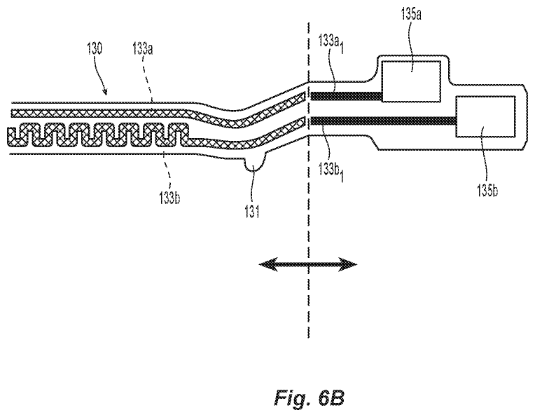

With reference to FIGS. 4 and 5, as noted above, jaw member 120 includes a structural frame 121, a jaw housing 122, and a tissue-treating plate 123 defining the tissue-treating surface 124 thereof. With reference also to FIG. 6A, details relating to the thermal cutter assembly 130 are generally defined to include the following elements (described internally to externally): substrate 131 or other internalized bendable metal structure that is both thermally and electrically conductive, e.g., stainless steel, aluminum, etc.; insulator 132 having generally electrically insulative properties and at least partially thermally conductive, e.g., sintered glass, alumina, plasma electrolytic oxidation (PEO anodize), Silica, etc.; resistive element 133 or any metal that is resistive but certain metals may have better thermal coefficients than others; and encapsulant 134 or an electrically insulative materials that is at least partially thermally conductive (may be the same or similar to the insulator). As explained below, the resistive element 133 may be deposited atop insulator 132 via sputtering or the like.

FIG. 6B shows a side view of thermal cutter assembly 130 and the electrical connections associated therewith. Generally, electrically conductive pads 135a, 135b connect to opposite ends 133a, 133b of resistive element 133 via traces 133a1, 133b1 which are electrically conductive traces (low resistance/low heat). As explained in detail below, resistive element 133 is configured to rapidly generate heat due to high resistive properties when electrical current is passed therethrough.

Structural frame 121 defines a proximal flange portion 126 and a distal body portion (not shown) extending distally from proximal flange portion 126. Proximal flange portion 126 is bifurcated to define a pair of spaced-apart proximal flange portion segments that receive proximal flange 111 of jaw member 110 therebetween and define aligned apertures 127 configured for receipt of pivot 103 therethrough/thereon to pivotably couple jaw members 110, 120 with one another (FIG. 5).

Jaw housing 122 of jaw member 120 is disposed about the distal body portion of structural frame 121, e.g., via overmolding, adhesion, mechanical engagement, etc., and supports tissue-treating plate 123 thereon, e.g., via overmolding, adhesion, mechanical engagement, depositing (such as, for example, via sputtering or thermal spraying), etc. Tissue-treating plate 123, as noted above, defines tissue-treating surface 124. Longitudinally-extending slot or channel 125 is defined through tissue-treating plate 123 and is positioned relative to jaw member 110 or an insulative member 115 disposed in vertical registration therewith when the jaw members 110 and 120 are in the approximated position (FIG. 5). The slot or channel 125 may be defined within an integrally-formed tissue-treating plate 123 or may be defined between two tissue-treating plates that, together, operate as a single treatment surface (not shown). Slot 125 may extend through at least a portion of jaw housing 122, a jaw insert (if so provided), and/or other components of jaw member 120 to enable receipt of thermal cutter assembly 130 at least partially within slot 125.

Thermal cutter assembly 130, more specifically, is disposed within longitudinally-extending slot 125 such that thermal cutter assembly 130 opposes jaw member 110 in the approximated position. Thermal cutter assembly 130 may be configured to contact jaw member 110 (or another insulative member 115 as mentioned above and as shown in FIG. 4) in the approximated position to regulate or contribute to regulation of a gap distance between tissue-treating surfaces 114, 124 in the approximated position. Alternatively or additionally, one or more stop members (not shown) associated with jaw member 110 and/or jaw member 120 may be provided to regulate the gap distance between tissue-treating surfaces 114, 124 in the approximated position.

Thermal cutter assembly 130 may be surrounded by the insulative member 115 disposed within slot 125 to electrically and/or thermally isolate thermal cuter assembly 130 from tissue-treating plate 123 (See FIG. 4 versus FIG. 5). As mentioned above, thermal cutter assembly 130 includes an encapsulant 134 that may act in conjunction with or in lieu of insulative member 115. Encapsulant 134 (and insulator 132 as shown in FIG. 6A) is configured cover the sides of the substrate 131 leaving the tissue facing edge 131a of the substrate exposed. Thermal cutter assembly 130 and insulative member 115 may similarly or differently be substantially (within manufacturing, material, and/or use tolerances) coplanar with tissue-treating surface 124, may protrude from tissue-treating surface 124, may be recessed relative to tissue-treating surface 124, or may include different portions that are coplanar, protruding, and/or recessed relative to tissue-treating surface 124.

Turning back to the thermal cutter assembly 130 and the various methods of manufacturing the same, it is contemplated that the resistive element 133 of the thermal cutter assembly 130 may be manufactured in thin layers that are deposited atop (or otherwise) insulator 132 which is disposed atop substrate 131. For the purposes herein, the resistive element 133 will be described as being deposited onto insulator 132, knowing that insulator, in turn, may be disposed on one or both sides of substrate 131. For example, it is contemplated that resistive element 133 may be deposited onto the insulator 132 via one or more of the following manufacturing techniques: sputtering, thermal evaporation, thermal spraying, cathodic arcing, pulsed laser deposition, electron beam deposition. Other techniques may include: electroless strike or plating and electro-plating, shadow masking.

Utilizing one or more of these techniques provides a thin layer of resistive material which has the benefit of dissipating heat quickly compared to a traditional thermal cutter assembly 130. Other advantages of thin-layered resistive elements 133 on the thermal cutter assembly 130 include: the ability to heat up quickly, the ability to require less energy to heat up and maintain heat during the cutting process, and the ability to cut tissue in a reduced timeframe compared to traditional electrical cutters.

Any one of the following materials (or combinations thereof) may be utilized as the resistive element 133: aluminum, copper, chromium, titanium, stainless steel, nickel, chrome, tin, platinum, palladium, gold, nichrome, and Kanthal®. It is contemplated that during manufacturing, combinations of materials may be utilized for a particular purpose or to achieve a particular result. For example, one material may be utilized as a base conductor with a second material used as an outer or inner conductor to act as the heating element. Additional techniques or materials may be added to act as thermal cutter assemblies 130 or resistive elements 133 such as those described with reference to U.S. patent application Ser. No. 16/785,347 filed Feb. 7, 2020, U.S. Provisional Patent Application Ser. No. 62/952,232 filed Dec. 21, 2019, U.S. patent application Ser. No. 16/838,551 filed Apr. 2, 2020, and U.S. patent application Ser. No. 16/518,016 filed Jul. 22, 2019, the entire contents of each of which being incorporated by reference herein.

In other embodiments, materials may be mixed during the application process. In some embodiments, the material used (e.g., Aluminum, copper etc.,) may be thin and still promote a good cutting effect while other materials may have to be thicker to produce the same or similar cutting effect due to the particular material's level of electrical resistance. In this latter instance, a highly conductive base material may be utilized with the thinner, less conductive material more resistive material to produce a desired effect.

In embodiments, a biocompatible material (not shown) may be utilized to cover a non-biocompatible material. In other embodiments, the materials may be deposited (or otherwise disposed on insulator 132 in non-uniform layers while still allowing for transitions, e.g., side-to-side transitions. The materials could be deposited (or otherwise disposed on insulator 132) in an alternating fashion and more than one electrical circuit may be employed.

Examples of resistive elements 133 that may be used for thermal cutter assemblies 130 may include single layer resistive elements 133 in the range of about 0.1 micron to about 500 microns. A so-called “thick” film resistive element 133 would be about 30 microns and a “thin” film resistive element 133 would be about 1 micron. Non-conductive, electrically transparent, thermally transparent, or electrically and/or thermally porous materials may also be layered in a similar fashion atop, below or between the resistive elements 133. One or more of these materials may be layered atop the resistive elements 133 to complete the thermal cutter assembly 130 as mentioned above within a specified range.

Generally, tissue-treating plates 113, 123 are formed from an electrically conductive material, e.g., for conducting electrical energy therebetween for treating tissue, although tissue-treating plates 113, 123 may alternatively be configured to conduct any suitable energy, e.g., thermal, microwave, light, ultrasonic, etc., through tissue grasped therebetween for energy-based tissue treatment. As mentioned above, tissue-treating plates 113, 123 are coupled to activation switch 80 and electrosurgical generator “G” (FIG. 1) such that energy may be selectively supplied to tissue-treating plates 113, 123 and conducted therebetween and through tissue disposed between jaw members 110, 120 to treat tissue, e.g., seal tissue on either side and extending across thermal cutter assembly 130.

Thermal cutter assembly 130, on the other hand, is configured to connect to electrosurgical generator “G” (FIG. 1) and second activation switch 90 to enable selective activation of the supply of energy to thermal cutter assembly 130 for heating resistive element 133 which, in turn, heats edge 131a of substrate 131 to thermally cut tissue disposed between jaw members 110, 120, e.g., to cut the sealed tissue into first and second sealed tissue portions. Other configurations including multi-mode switches, other separate switches, etc. may alternatively be provided.

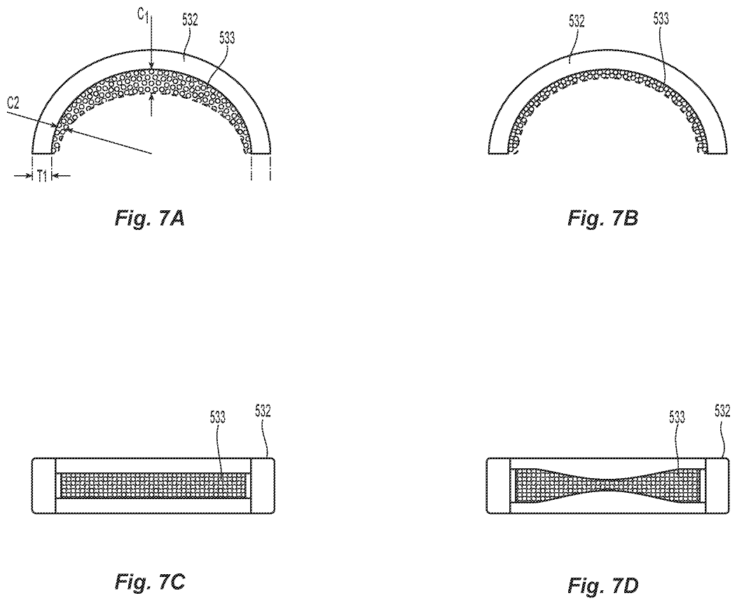

As shown in FIGS. 7A-7D, in embodiments where end effector assembly 100, or a portion thereof, is curved, longitudinally-extending slot 125 and thermal cutter assembly 130 of jaw member 120 may similarly be curved, e.g., wherein longitudinally-extending slot 125 and thermal cutter assembly 130 (or corresponding portions thereof) are relatively configured with reference to an arc (or arcs) of curvature rather than a longitudinal axis. If end effector 100 is curved, longitudinally-extending slot 125 and thermal cutter assembly 130 may also remain straight or vice versa. Thus, the terms longitudinal, transverse, and the like as utilized herein are not limited to linear configurations, e.g., along linear axes, but apply equally to curved configurations, e.g., along arcs of curvature. In such curved configurations, jaw member 110 and any contemplated features thereof are likewise curved.

Referring particularly to manufacturing a curved thermal cutter assembly 130 that is configured to follow a curved jaw member, e.g., jaw member 120, typical deposition techniques, e.g., sputtering, used with depositing conductive material onto straight insulators 132 (or insulators 132 and substrates 131), may result in non-uniform thickness along the thermal cutter assembly 130. More particularly, when depositing a conductive material onto a flat substrate (e.g., insulator 132), layering is well controlled and temperatures across the thermal cutter assembly 130 during activation remain relatively consistent. Moreover, only a 2-axis dispensing machine is required for deposition. When trying to manufacture a curved cutting element, e.g., thermal cutter assembly 130, once the resistive element 133 is deposited, one technique would be to bend the thermal cutter assembly 130 into the desired curve to fit within the longitudinal slot 125 defined within jaw member 120. However, the stress of the bending of the thermal cutter assembly 130 (either the substrate 131, insulator 132 or deposited resistive element 133) after deposition may not yield consistent results and may cause temperature variants along the thermal cutter assembly 130.

One solution would be to bend the substrate 131 and insulator 132 prior to deposition or use a pre-curved substrate 131 and insulator 132. However, bending the substrate 131 and insulator 132 prior to deposition (e.g., in particular, sputtering) similarly may not yield consistent results. More particularly, bending the substrate 131 and insulator 132 and then depositing a conductive material of the resistive element 133 thereon may result in a greater build-up of conductive material towards the center of the curve of the substrate 131 and insulator 132 and a thinner amount of conductive material of the resistive element 133 towards the outer peripheral surfaces thereof which, again, may cause inconsistent temperature gradients along the thermal cutter assembly 130.

FIGS. 7A and 7B show one envisioned thermal cutter assembly 530 and manufacturing technique associated with the same wherein the thickness conductive material of the resistive element 533 is varied along a length, height or a width of the substrate 532 to compensate for possible inconsistencies in the deposition process as a result of the curvature thereof. More particularly, substrate 531 is initially bent (or pre-bent) during assembly and then the resistive element 533 is deposited thereon. Substrates 531 that can handle the stress of deformation (e.g., bending) may be utilized to reduce possible manufacturing and performance issues.

During the deposition process, e.g., in particular, sputtering, the conductive material of the resistive element 533 is deposited on the substrate 532 along a length thereof. The nature of the deposition process on a curved surface causes the conductive material of the resistive element 533 to vary in thickness, e.g., a thickness of C1 at the center, a lesser thickness of C2 at the outer peripheral surfaces and variable therebetween (FIG. 7A).

As a result of the varying thickness of the resistive element 533 along the length of the substrate 532, the exposed edge of the substrate 532 may heat in a non-uniform manner. By tailoring the width of the conductive material of the resistive element 533 during the deposition process along the substrate 532 more uniform heating of the thermal cutter assembly 530 may be achieved. For example, and as shown in FIG. 7D, to compensate for the varying thickness of the conductive material shown in FIG. 7A above, the center of the resistive element 533 is thinner in height and wider as the resistive element 533 extends to the proximal and distal ends thereof along the substrate 532 such that the cross-sectional area along the resistive element 533 remains substantially consistent resulting in uniform heating.

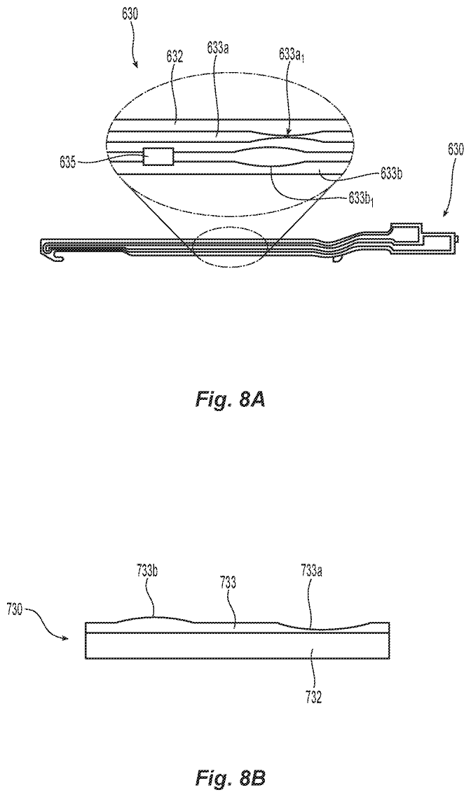

FIGS. 8A and 8B show additional examples of the resistive elements 633 disposed along insulators 632 and substrates 631 wherein the geometry of the resistive element 633 is changed to provided uniform heating along the length thereof. More particularly, FIG. 8A shows two resistive elements or traces 633a, 633b disposed atop insulator 632 and extending therealong. Resistive element 633a includes an area 633a1 with a reduced width which is configured to compensate for a cold spot along the thermal cutter assembly 630 and resistive element 633b includes an area 633b1 with an expanded width which is configured to compensate for a hot spot along the thermal cutter assembly 630. Adding a low resistance pad 635 over the restive element 633b will also compensate for a hot spot as the resistive element 633b will run cooler.

FIG. 8B shows a side view of thermal cutter assembly 730 having a restive element 733 with differing geometries to affect the temperature thereof. For example, resistive element 733 at thin area 733a will compensate for a cold spot as the resistive element 733 in that area 733a will run hotter and resistive element 733 at thicker area 733b will compensate for a hot spot as the resistive element 733 in that thicker area 733b will run cooler.

As can be appreciated, a more complex dispensing machine (not shown) may be required and the final dimensions of the thermal cutter assemblies described herein may need to be configured to meet the profile of the cutter assembly slot, e.g., slot 125, defined within the jaw member 120. Moreover, since the resulting thickness of the conductive material or the resistive element is not as finely controlled as with deposition techniques onto flat surfaces, the temperature of the thermal cutter assemblies described herein may not be as well-controlled.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

What is claimed is:

1. A jaw member for a surgical instrument, comprising:
a curved jaw housing supporting a similarly curved electrically conductive tissue treating surface having slot defined therein and extending therealong, the slot defining a curve substantially parallel to the curve of the tissue treating surface; and
a thermal cutter assembly disposed within at least a portion of the slot, the thermal cutter assembly including a curved substrate configured to support a resistive element deposited thereon, the resistive element adapted to connect to an energy source and configured to thermally conduct heat to a portion of the substrate exposed from within the slot, the resistive element composed of a conductive material, a thickness of the conductive material being varied along a length of the resistive element to compensate for inconsistencies in thermal heating as a result of a deposition process.

2. The jaw member according to claim 1, wherein the conductive material is selected from the group consisting of aluminum, copper, chromium, titanium, stainless steel, nickel, chrome, tin, platinum, zinc, palladium, gold, nichrome, and ferritic iron-chromium-aluminum alloys.

3. The jaw member according to claim 1, wherein the conductive material includes a thickness in the range of about 0.1 micron to about 500 microns along the length of the substrate.

4. The jaw member according to claim 1, wherein the conductive material includes multiple materials selected from the group consisting of aluminum, copper, chromium, titanium, stainless steel, nickel, chrome, tin, platinum, zinc, palladium, gold, nichrome, and ferritic iron-chromium-aluminum alloys, the total thickness of the multiple materials having a thickness in the range of about 1 micron to about 30 microns.

5. The jaw member according to claim 1, wherein the conductive material includes a first thickness proximate a center of the substrate and a second thickness proximate an outer periphery of the substrate.

6. The jaw member according to claim 5, wherein the first thickness of the conductive material is less than the second thickness of the conductive material.

7. The jaw member according to claim 5, wherein the first thickness of the conductive material is greater than the second thickness of the conductive material.

8. The jaw member according to claim 1, wherein the conductive material includes a first thickness proximate a center of the substrate and a second thickness proximate the proximal and distal ends of the substrate wherein the cross-sectional area of the resistive element along the substrate is substantially consistent resulting in uniform heating of the thermal cutting assembly at any point along a length thereof.

9. The jaw member according to claim 1, wherein the deposition process includes at least one of sputtering, thermal evaporation, thermal spraying, cathodic arcing, pulsed laser deposition, electron beam deposition, electroless strike, electro-plating, or shadow masking.

10. A jaw member for a surgical instrument, comprising:
a curved jaw housing supporting a similarly curved electrically conductive tissue treating surface having slot defined therein and extending therealong, the slot defining a curve substantially parallel to the curve of the tissue treating surface; and
a thermal cutter assembly disposed within at least a portion of the slot, the thermal cutter assembly including a curved substrate configured to support an insulator thereon, the insulator, in turn, configured to receive a resistive element deposited thereon, the resistive element encapsulated by an encapsulant, adapted to connect to an energy source and configured to thermally conduct heat to a portion of the substrate exposed from within the slot, the resistive element composed of a conductive material, a thickness of the conductive material being varied along a length of the resistive element to compensate for inconsistencies in thermal heating as a result of a deposition process, wherein the cross-sectional area of the resistive element along the substrate is substantially consistent resulting in uniform heating of the thermal cutting assembly at any point along a length thereof.

11. The jaw member according to claim 10, wherein the conductive material is selected from the group consisting of aluminum, copper, chromium, titanium, stainless steel, nickel, chrome, tin, platinum, zinc, palladium, gold, nichrome, and ferritic iron-chromium-aluminum alloys.

12. The jaw member according to claim 10, wherein the conductive material includes a thickness in the range of about 0.1 micron to about 500 microns along the length of the substrate.

13. The jaw member according to claim 10, wherein the conductive material includes a first thickness proximate a center of the substrate and a second thickness proximate an outer periphery of the substrate.

14. The jaw member according to claim 10, wherein the encapsulant is at least partially thermally conductive and is also disposed on an opposite side of the substrate.

15. A method for manufacturing a thermal cutter assembly for use with a curved jaw member of a surgical instrument, comprising:
disposing an insulator atop a curved substrate along a length thereof;
depositing a conductive material atop the insulator along a length thereof to form a resistive element and varying the thickness of the conductive material along the curve to ensure a uniform temperature gradient of the resistive element when activated along the length thereof; and
encapsulating the resistive element with an encapsulant.

16. A method for manufacturing a thermal cutting assembly for use with a jaw member of a surgical instrument according to claim 15, wherein the deposition process includes at least one of sputtering, thermal evaporation, thermal spraying, cathodic arcing, pulsed laser deposition, electron beam deposition, electroless strike, electro-plating, or shadow masking.

17. A method for manufacturing a thermal cutting assembly for use with a jaw member of a surgical instrument according to claim 15, wherein the conductive material is selected from the group consisting of aluminum, copper, chromium, titanium, stainless steel, nickel, chrome, tin, platinum, zinc, palladium, gold, nichrome, and ferritic iron-chromium-aluminum alloys.

18. A method for manufacturing a thermal cutting assembly for use with a jaw member of a surgical instrument according to claim 15, wherein the conductive material includes a first thickness proximate a center of the substrate and a second thickness proximate an outer periphery of the substrate.

19. A method for manufacturing a thermal cutting assembly for use with a jaw member of a surgical instrument according to claim 15, further comprising encapsulating an opposite side of the substrate with the encapsulant.

20. A method for manufacturing a thermal cutting assembly for use with a jaw member of a surgical instrument according to claim 19, wherein the encapsulant is at least partially thermally conductive.
